(19) United States
(12) Patent Application Publication
(10) Pub. No.: US 2025/0281707 A1
(43) Pub. Date: Sep. 11, 2025

(54) SYSTEMS AND METHODS FOR PREDICTING EXTUBATION READINESS

(71) Applicant: University of Kentucky Research Foundation, Lexington, KY (US)

(72) Inventors: Elie G. Abu Jawdeh, Southlake, TX (US); Mandy P. Brasher, Farmers Branch, TX (US); Alexandr Virodov, Lexington, KY (US)

(21) Appl. No.: 19/074,111

(22) Filed: Mar. 7, 2025

Related U.S. Application Data
(60) Provisional application No. 63/563,677, filed on Mar. 11, 2024.

(51) Int. Cl. A61M 16/00; G16H 10/60; G16H 50/20

(52) U.S. Cl. CPC A61M 16/026; A61M 16/0003; G16H 10/60; G16H 50/20; A61M 16/0051; A61M 2205/18; A61M 2205/3303; A61M 2205/3306; A61M 2205/3327; A61M 2205/3331; A61M 2205/3379; A61M 2205/502; A61M 2230/06; A61M 2230/20; A61M 2230/46; A61M 2240/00

(57) ABSTRACT

Embodiments provided herein include systems and methods for determining extubation readiness. One embodiment of a method includes obtaining pulse oximeter data and ventilator data for a patient that has been intubated over a predetermined monitoring period, classifying a lung disease state of the patient as acute or chronic based on at least one of the following: an age of the patient or a clinical indicator, and providing the pulse oximeter data, the ventilator data, and a lung disease classification to a trained predictive model previously trained on training data. Some embodiments include calculating a likelihood of extubation success or failure within a clinically relevant time window, and generating at least one of the following: a positive predictive value (PPV) or a negative predictive value (NPV) and generating and presenting, by the computing device, a readiness output based on the likelihood of extubation success or failure for determining extubation timing.

### CROSS REFERENCE

This application claims the benefit of U.S. Provisional Application Ser. No. 63/563,677, filed Mar. 11, 2024, which is hereby incorporated by reference in its entirety.

### GOVERNMENT SUPPORT

This invention was made with support from the National Institutes of Health National Institute of Child Health and Human Development under grant K23 HD109471-01A1 and the National Institutes of Health National Center for Advancing Translational Services under grant UL1TR001998. The government may have certain rights to the invention.

### TECHNICAL FIELD

Embodiments described herein generally relate to systems and methods for predicting extubation readiness in patients, such as infants, preterm infants, etc. using machine-learning analysis of outputs from one or more medical devices, such as bedside pulse oximeter, ventilator data, etc.

### BACKGROUND

As background, intubation and mechanical ventilation are commonly utilized for preterm infants with respiratory failure. However, prolonged mechanical ventilation is associated with the neonatal morbidities of bronchopulmonary dysplasia, pulmonary hypertension, severe retinopathy of prematurity, and periventricular leukomalacia. Moreover, in cases of failed extubation attempts, infants encounter complications of reintubation and setbacks in respiratory status.

More specifically, there are currently a limited number of accurate tools to assist clinicians in assessing extubation readiness. Analysis of many factors such as demographics, laboratory values, ventilator settings, and quantification of respiratory activity have been attempted with limited utility. Tests for extubation readiness, such as spontaneous breathing trials, have limited use. The lack of validated objective extubation criteria for preterm infants is also reflected in the high level of practice variation among institutions for weaning and extubation protocols.

Mechanical ventilation is common in many types of patients, such as preterm infants, infants, older patients, etc. in the treatment of respiratory distress and respiratory failure. Although essential and lifesaving, prolonged mechanical ventilation is associated with increased morbidities. Nevertheless, untimely extubation may also be harmful, as failure and subsequent reintubation is associated with increased morbidity and mortality as well. Therefore, a timely and safe extubation should be undertaken to shorten the duration of mechanical ventilation after the resolution of respiratory distress or failure.

However, there are no standardized processes to assess for extubation readiness and marked variation among neonatal intensive care units (NICUs). Multiple strategies have been investigated, such as use of spontaneous breathing tests, formal pulmonary function testing, blood gases, and diverse demographics, all with variable success. The utility of the aforementioned predictors is inconsistent or of limited availability at the bedside. Consequently, an objective, feasible, and readily available assessment for extubation readiness is yet to be determined.

Therefore, a need exists for an optimized assessment method for determining extubation readiness for various patient populations in order to achieve improved outcomes.

### SUMMARY

Embodiments provided herein include systems and methods for determining extubation readiness. One embodiment of a method includes obtaining pulse oximeter data and ventilator data for a patient that has been intubated over a predetermined monitoring period, classifying a lung disease state of the patient as acute or chronic based on at least one of the following: an age of the patient or a clinical indicator, and providing the pulse oximeter data, the ventilator data, and a lung disease classification to a trained predictive model previously trained on training data. Some embodiments include calculating a likelihood of extubation success or failure within a clinically relevant time window, and generating at least one of the following: a positive predictive value (PPV) or a negative predictive value (NPV) and generating and presenting, by the computing device, a readiness output based on the likelihood of extubation success or failure for determining extubation timing.

Embodiments of a system include an oximeter for measuring an oxygen level of a patient, a ventilator for intubating and ventilating the patient, and a computing device. The computing device may include a memory component and logic. The logic may be executed by the computing device to cause the system to obtain pulse oximeter data from the oximeter and ventilator data from the ventilator for the patient that has been intubated over a predetermined monitoring period, classify a lung disease state of the patient as acute or chronic based on at least one of the following: an age of the patient or a clinical indicator, and provide the pulse oximeter data, the ventilator data, and a lung disease classification to a trained predictive model previously trained on training data. Some embodiments include calculating a likelihood of extubation success or failure within a clinically relevant time window, and generating at least one of the following: a positive predictive value (PPV) or a negative predictive value (NPV) and generating and presenting a readiness output based on the likelihood of extubation success or failure for determining extubation timing.

Some embodiments include a medical device for predicting extubation readiness for a patient. Embodiments of the medical device includes a display and stores logic that, when executed by the medical device, causes the medical device to obtain pulse oximeter data and ventilator data for a patient that has been intubated over a predetermined monitoring period, classify a lung disease state of the patient as acute or chronic based on at least one of the following: an age of the patient or a clinical indicator, and provide the pulse oximeter data, the ventilator data, and a lung disease classification to a trained predictive model previously trained on training data. Some embodiments cause the medical device to calculate a likelihood of extubation success or failure within a clinically relevant time window, and generating at least one of the following: a positive predictive value (PPV) or a negative predictive value (NPV) and generate and present a readiness output via the display based on the likelihood of extubation success or failure for determining extubation timing.

### BRIEF DESCRIPTION OF THE DRAWINGS

The embodiments set forth in the drawings are illustrative and exemplary in nature and not intended to limit the disclosure. The following detailed description of the illustrative embodiments can be understood when read in conjunction with the following drawings, where like structure is indicated with like reference numerals and in which:

FIG. 1 depicts a computing environment for predicting extubation readiness, according to one or more embodiments shown and described herein;

FIG. 2A depicts a graphical representation of extubation success relative to a percentage of time a patient experiences hypoxemia, according to one or more embodiments shown and described herein;

FIG. 2B depicts a graphical representation of extubation success relative to a frequency of intermittent hypoxemia per day, according to one or more embodiments shown and described herein;

FIG. 3A depicts a graphical representation of a receiver operating characteristic curve of an extubation event by various models, according to one or more embodiments shown and described herein;

FIG. 3B depicts a graphical representation of a receiver operating characteristic curve of an extubation event by various models, according to one or more embodiments shown and described herein;

FIG. 3C depicts a graphical representation of a receiver operating characteristic curve of an extubation event by various models, according to one or more embodiment shown and described herein;

FIG. 4 depicts a flowchart for predicting extubation readiness in preterm infants, according to embodiments provided herein; and

FIG. 5 depicts a computing device for predicting extubation readiness in preterm infants, according to embodiments provided herein.

### DETAILED DESCRIPTION

Provided herein are systems and methods for predicting extubation readiness in patients, such as preterm infants, children, adolescence, elderly patients, mammals, etc. using machine-learning analyses, statistical analyses, and/or other analyses such as deep learning artificial intelligence models of bedside pulse oximeter (for measuring an oxygen level and/or other data) and ventilator data. Some embodiments may be configured to enhance a prediction accuracy of extubation readiness in patients, such as preterm infants, while utilizing readily available data streams from medical devices, such as bedside pulse oximeters, monitors, and/or ventilators. Moreover, the assessment of success or failure may include extubation readiness and/or reintubation readiness for high-risk patient populations may allow for improved outcomes, as will be described in additional detail herein.

In some embodiments, success and/or failure may be defined by a medical provider such as via a medical device and/or other computing device. As an example, a first medical facility may define success, based on a first plurality of predetermined factors. A second medical facility may define success based on a second plurality of predetermined factors. Some embodiments may define failure based on a third plurality of predetermined factors.

Pulse oximetry is commonly used in clinical practice, with adapted capabilities to calculate and display cumulative intermittent hypoxemia (IH), sometimes called intermittent hypoxia, in the form of oxygen saturation (SpO2) histograms. Intermittent hypoxemia is underutilized in assessing extubation readiness, since IH is the result of both lung disease and respiratory instability in patients. The disclosed system and methods determine the value of IH in extubation successes and failures in those patients. Moreover, the disclosed system and method utilizes machine-learning analyses, statistical analyses, and/or other analyses as described above to develop a practical and more accurate model for prediction of extubation readiness in patients by analyzing streams of data available and using bedside pulse oximeters and/or ventilators.

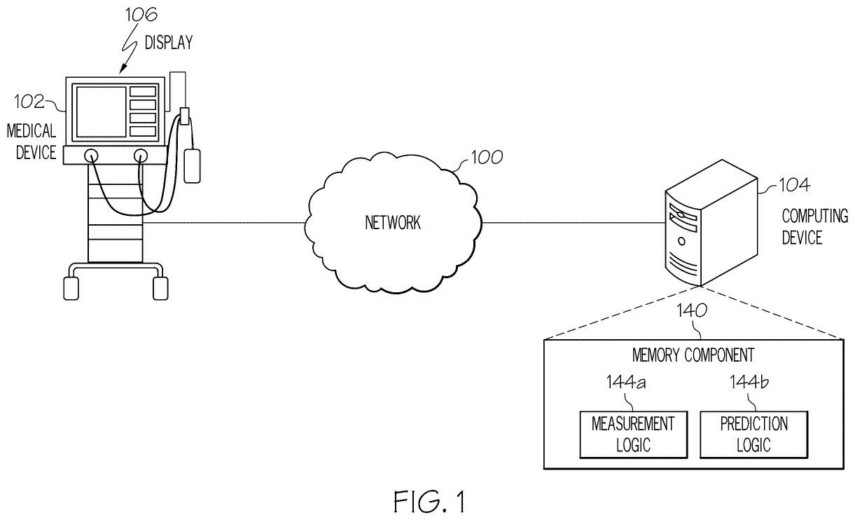

Referring now to the drawings, FIG. 1 depicts a computing environment for predicting extubation readiness, according to one or more embodiments shown and described herein. As illustrated, a network 100 may be coupled to a medical device 102 (one or more) and a computing device 104. The network 100 may include one or more wide area network (WAN), local area network (LAN), and/or personal area network (PAN). Example WANs might include the internet, a WiMax network, a cellular network, a public switched telephone network (PSTN), and/or the like. Example LANs might include an Ethernet network, a wireless fidelity (Wi-Fi) network, and/or the like. Example PANs might include Bluetooth, Zigbee, and/or other peer-to-peer networks.

Coupled to the network 100 is the medical device 102. The medical device 102 may be configured as any medical device that may be utilized for intubating and ventilating a patient, measuring oxygen saturation indicative of intermittent hypoxemia events, heart rate, perfusion, ventilator data, such as tidal volume, airway pressure, and a ventilation mode, and postnatal age and corresponding disease classification, etc. The medical device 102 may include other monitoring devices that monitor blood pressure, chest wall excursion, diaphragm contraction, carbon dioxide levels, etc.

As an example, the medical device 102 may include a bedside ventilator (e.g., Draeger Babylog VN500, etc.), which may provide one or more metric variables, including ventilator settings (e.g., mode, set pressure, set tidal volumes, mandatory rate, pressure support, positive end expiratory pressure (PEEP), inspiratory to expiratory ratio, and FiO₂) as well as variables measured by the ventilator (e.g., measure pressure, measured volume, spontaneous tidal volumes, resistance, and compliance). As such, the medical device 102 may include medical instruments for coupling with a patient, as well as computing components, such as a memory component, processor, logic, etc. for understanding the signals received from the patient and communicating the data to the computing device 104. The medical device 102 may also include a display 106, which may be configured as a monitor, speaker, and/or other output device for communicating data to a user.

As an example, the display 106 may be configured for providing output of data, including an indicator for a high probability of extubation success, an indicator for an intermediate or uncertain probability, an indicator for a high probability of extubation failure, etc. Similarly, the display 106 may also provide a reintubation alert and/or extubation alert to indicate when a patient is to be intubated, reintubated, and/or extubated. Some embodiments may be configured to determine and/or predict differing probabilities of extubation success and failures and present a readiness output corresponding to the differing probabilities of extubation success via the display 106. Regardless, some embodiments may combine the medical device 102 and the computing device 104 for providing the described functionality with a single device and/or a plurality of devices. As an example, the display device 106 is depicted in FIG. 1 as being integral with the medical device 102. This is but one embodiment. Some embodiments may be configured to provide output from the medical device via a different device altogether, such as by computing device 104, a mobile device (not explicitly depicted in FIG. 1), etc.

The computing device 104 may also be coupled to the network 100. The computing device 104 may be configured as a personal computer, laptop, mobile device, or other computing device and may be local or remote. The computing device 104 may be accessed by the medical device 102 and thus may receive baseline data related to parameters that are considered normal or problematic for various conditions of patients. Similarly, the computing device 104 may receive measured data, which represents data received from the medical device 102.

The computing device 104 may include a memory component 140 that stores measurement logic 144a and prediction logic 144b. As described in more detail below, the measurement logic 144a and the prediction logic 144b represent software that is executed by the computing device 104, but may be implemented by one or more different pieces of software. That being said, in the example of FIG. 1, the measurement logic 144a may be configured to cause the computing device 104 to receive and interpret measurement data received from the medical device 102. The prediction logic 144b may include a machine-learning module and/or other analyses models in some embodiments and may be configured to cause the computing device 104 to utilize the measured data and predict a success likelihood or a failure likelihood in intubation or extubation of a patient, as described in more detail below.

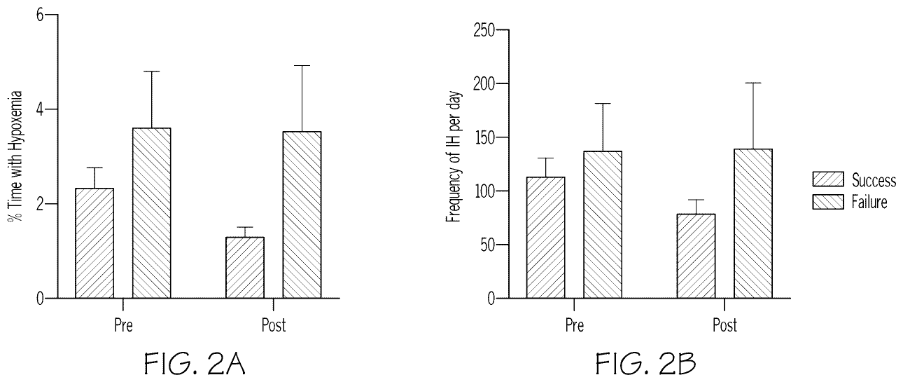

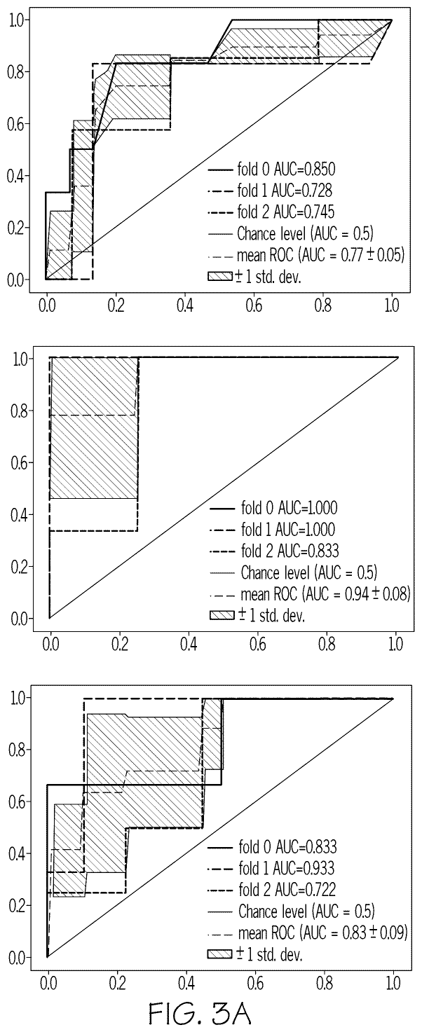

FIGS. 2A and 2B, in developing the disclosed embodiments, patients, such as infants having less than 30 weeks gestational age (GA), elderly patients, etc. may be monitored upon admission to the medical facility. These embodiments involve initially obtaining SpO2 data (sampling interval: 1 Hz, averaging time: 2 seconds). Other embodiments may utilize different sampling intervals and/or metrics.

These embodiments further involve collecting respiratory support and extubation data from patient medical records. For purposes of the present disclosure, IH may be calculated as percent time spent with hypoxemia (SpO2<80%) and number of events per day when oxygen saturation falls below 80%. However, it should be appreciated that other predetermined thresholds of SpO2 or measures (IH profile) and/or changes in SpO2 from baseline, may be utilized, as has been described herein. In analyzing extubation readiness, IH measures may be reported pre-extubation until post-extubation (or when reintubation or first intubation became necessary). In some embodiments, the thresholds may include one or more of the following: SpO2<75%, SpO2<85%, SpO2<90%, deviation by 1 to >5% from baseline or from baseline or fluctuation into hyperoxemia (also known hyperoxia). Similarly, some embodiments may include heart rate thresholds, such as heart rate <100 beats per minute, 80 beats per minute, <⅔ baseline, >160 beats per minute, >190 beats per minute, etc. Some embodiments may utilize thresholds for perfusion changes, and/or other criteria.

Embodiments provided herein further involve conducting statistical analyses of the obtained data described herein. For example, group comparisons of mean values for the percent time (% time IH— SpO2<80) and the number of events (IH— SpO2<80) may be square root transformed, if necessary, in order to meet statistical assumptions as previously described. Some embodiments may define failure as re-intubation before 72 hours post-extubation. Some embodiments may provide a different timeline, for example, within 24 or 48 hours or within 2 weeks, and/or different criteria for defining failure, for example, increase in oxygen requirement >40%, acidotic blood pH samples, or hypercarbia. Receiver operating characteristic (ROC) curve analyses may be may be used to find optimal cutoff values. Secondary analyses may examine differences between groups at 24-hour post-extubation. All tests may be two-sided at the 5% significance level. Some embodiments may utilize duration of post-extubation. Pre-extubation and post-extubation may be examined, for example any duration between one and three days.

Referring still to FIGS. 2A and 2B, initial results are depicted. In these embodiments, the test group may include median GAs from 26-6/7 weeks and 25-5/7 weeks in success and failure groups, respectively. All infants may be extubated from conventional ventilator support to continuous positive airway pressure (CPAP) or noninvasive positive-pressure ventilation (NIPPV). Overall, most extubation attempts may be successful (72% of events may be successful at 72 hours).

As further depicted in FIGS. 2A and 2B, continuous SpO2 waveforms may be interrogated for IH events and SpO2 histograms before and after extubation. Differences in IH measures between failure and success groups are further depicted in FIGS. 2A and 2B. For example, both % time—SpO2<80 and IH— SpO2<80 may be higher in the failure group compared to successful group pre-extubation and post-extubation, however, differences may be not statistically significant. On secondary analyses for differences in 24 hour post-extubation, there may be also increased IH measures in the failure group compared to the success group (% time—SpO2<80, p=0.07, and IH— SpO2<80, p=0.03). Furthermore a statistically significant decrease exists in IH after extubation in the success group in both the primary (72 hours) and secondary (24 hours) analyses (all p<0.01) (FIG. 2A). The differences in IH measures remained the same after adjusting for GA, birth weight, weight, and day of life at time of extubation.

Furthermore, Demographics and respiratory support data related to FIGS. 2A and 2B are presented in Table 1 below:

TABLE 1
Demographics and respiratory characteristics

| | Success | | Failure | |
|---|---|---|---|---|
| Baseline Characteristics | n = 49 | | n = 19 | |
| Gestational age (weeks) | 26.6 | (25.3-27.6) | 25.5 | (25.1-26.1) |
| Birth weight (grams) | 800 | (730-1040) | 730 | (650-905) |
| Weight at time of extubation (grams) | 1140 | (960-1253) | 970 | (830-1150) |
| Age at time of extubation (days) | 18 | (5-37) | 21 | (9-33) |
| Baseline Ventilation Setting | | | | |
| Set respiratory rate (breaths/min) | 15 | (15-20) | 20 | (15-20) |
| FiO₂ (%) | 25 | (21-30) | 29 | (25-32) |
| PEEP (cmH₂O) | 6 | (5-6) | 6 | (6-7) |
| PIP (cmH₂O) | 15 | (13-21) | 16 | (13-18) |
| TV (mL/kg) | 5 | (5-6) | 5 | (4-6) |
| Post-extubatation non-invasive support | | | | |
| CPAP | 10/49 | (20%) | 0/19 | (0%) |
| NIPPV | 39/49 | (79%) | 19/19 | (100%) |
| FiO₂ (%) | 32 | (25-38) | 40 | (30-44) |
| PEEP (cmH₂O) | 7 | (6-8) | 8 | (7-9) |

Median (interquartile range).
FiO₂: fraction of inspired oxygen.
PEEP: positive end expiratory pressure.
PIP: peak inspiratory pressure.
TV: Tidal volume.
CPAP: Continuous positive airway pressure.
NIPPV: Noninvasive positive-pressure ventilation.

In view of the foregoing, it should be appreciated that the disclosed embodiments identify a potential and valuable role for IH in extubation success or failure in preterm infants. For example, there may be a trend for increased IH pre-extubation in the failure group as compared to the success group. Second, the same trend between failure and success groups may occur post-extubation, indicating a potential role of IH in early detection of impending failures and prompt re-intubation or to guide the titration of non-invasive respiratory support (increasing support in those likely to fail and weaning promptly in those showing signs of success).

Further, it is understood that infants may continue to have frequent IH events during mechanical ventilation. However, as depicted in FIGS. 2A and 2B, it is illustrated that infants who had successful extubations had a subsequent significant decrease in IH measures post-extubation. This may be useful for early identification of extubation success versus failure and guide management of non-invasive respiratory support.

In some embodiments described herein, IH may also be used as a marker for both timely identification of extubation readiness and impending failure, lending to safe reinsertion of the endotracheal tube. The value of IH, as derived from the SpO2 histogram of FIGS. 2A and 2B, is both readily available in all NICUs and often an accurate consequence of a cardio-respiratory compromise. Limitations include the single-center retrospective nature of this study and limited power. In addition, while NICU criteria for extubation are in place, the final decision to extubate, or the need to reintubate remained at the discretion of the treating clinical team.

Referring still to FIGS. 2A and 2B, it should be further appreciated that utilization of the bedside SpO2 histogram for cumulative IH as a predictor for extubation readiness may be used to determine a time to extubate infants recovering from respiratory distress.

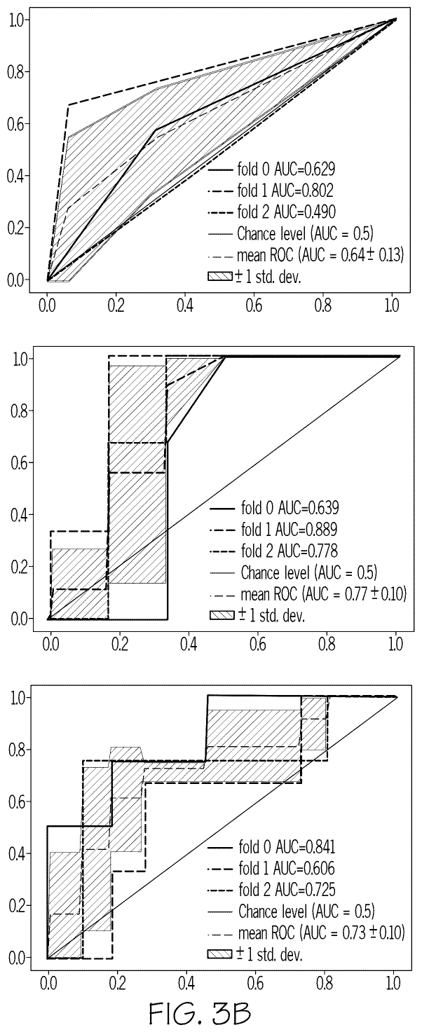

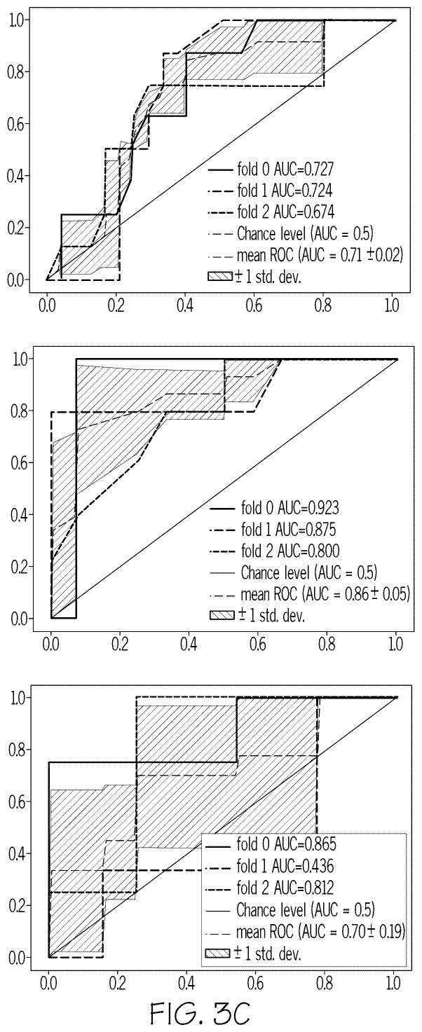

Turning now to FIGS. 3A-3C, it should be appreciated that some embodiments may be implemented to determine extubation readiness of an infant. For example, these embodiments may involve analyzing infants born at <30 weeks gestational age (GA) who had an extubation event during their neonatal intensive care unit (NICU). As such, these embodiments may involve obtaining high-resolution oxygen saturation (SpO₂) data (Masimo Radical 7, 1 Hz sampling interval, 2-second averaging time). While some embodiments may utilize high resolution measurements, some embodiments may use lower resolution measurements. Further, these embodiments may involve processing high-resolution pulse oximeter data to accurately quantify intermittent hypoxemia (IH) at the predetermined threshold of SpO₂<80%. In some embodiments, the thresholds may include one or more of the following: SpO₂<75%, SpO₂<85%, SpO₂<90%, deviation by 1 to >5% from baseline or fluctuation into hyperoxemia (also known hyperoxia). Similarly, some embodiments may include heart rate thresholds, such as heart rate <100 beats per minute, 80 beats per minute, <⅔ baseline, or increase in heart rate, etc. Some embodiments may utilize thresholds for perfusion changes, and/or other criteria. This may include the total fraction of time spent below the predetermined threshold of SpO₂ and number of events when SpO₂ levels fell below the predetermined threshold in any given time interval.

These embodiments may further involve collecting ventilator metrics from bedside ventilators (Draeger Babylog VN500; 4-5 min sampling, or other similar sampling intervals or other ventilator or frequency of data collection). This may include collecting a number of metric variables, including ventilator settings (e.g., mode, set pressure, set tidal volumes, mandatory rate, pressure support, positive end expiratory pressure (PEEP), inspiratory to expiratory ratio, and fraction of inspired oxygen (FiO₂)) as well as variables measured by the ventilator (e.g., measure pressure, measured volume, spontaneous tidal volumes, resistance, and compliance).

Some embodiments may include a user, administrator, medical provider, etc. selecting one or more predictive interval (seconds, minutes, hours, or days) within a monitoring period. For example, some embodiments may specify one or more of the following: 30 seconds, 30 minutes, 1 hour, 2 hours, 5 hours, 1-2 days, etc.

In some embodiments, one extubation event per infant may be analyzed. Events analyzed may be first time or subsequent extubation attempts. The machine-learning models employed may be further designed to predict extubation failure, which, as described herein, may be defined as the need for reintubation within a predetermined amount of time from extubation or defined by other thresholds. To separate the differing pathology of the acute postnatal lung and the subacute lung or chronic lung disease condition of older infants, an analysis may partition infants by a predetermined postnatal age threshold at <2 weeks or >2 weeks or other age threshold. However, it should be appreciated that other partitions based on gestational age, corrected age, and/or postnatal age may be utilized. These embodiments may be used to predict extubation or respiratory management based on types of lung pathology, such as in cases of acute versus chronic lung disease, in cases of restrictive versus obstructive lung disease, in cases of congenital cardiac pulmonary disease, etc.

These embodiments may further involve developing a number of model sets using the obtained data and the machine-learning models. For example, three model sets may be developed using different data streams. The first model may use a combination of pulse oximeter and ventilator data (IH+SIMV). The other two models may include pulse oximeter only (IH) or ventilator (SIMV) only data. Depending on the particular embodiment, data provided by pulse oximeters may include SpO2, heart rate, perfusion, hemoglobin, carbon dioxide, etc.). Similarly, a ventilator may include carbon dioxide. Similarly, demographic birth information and data from time of extubation (e.g., age, weight, sex, race, species, past medical history or diagnoses, etc.) may be included in all analyses. For example, the table below depicts the demographic information of the extubation events analyzed herein:

TABLE 1
Demographic Information and Number of Extubation Events Analyzed

| | All | | Success | | Failure | |
|---|---|---|---|---|---|---|
| Extubation events (n) | 110 | | 84 | | 26 | |
| Gestational age at birth (weeks) | 26.0 | (25.1-27.0) | 26.1 | (25.1-27.5) | 25.6 | (24.6-26.1) |
| Age at extubation (days) | 12.1 | (2.1-35.6) | 12.2 | (2.0-38.4) | 10.6 | (1.8-29.2) |
| Birth Weight (g) | 825 | (678-968) | 865 | (700-1045) | 865 | (650-860) |
| Weight at Extubation (g) | 1075 | (869-1240) | 1140 | (945-1260) | 895 | (795-1070) |

| | | | |
|---|---|---|---|
| Female (%) | 53.6 | 60.7 | 30.8 |
| Black (%) | 11.8 | 10.7 | 15.4 |
| White, Hispanic (%) | 0.9 | 1.2 | 0 |
| White, not Hispanic (%) | 87.3 | 88.1 | 84.6 |

Median (interquartile range).

As previously described herein, variables from both the pulse oximeter and ventilator may be input based on computed percentiles calculated from the data set. Percentiles may be separated into quantiles defined at 10%, 25%, 50% (median), 75%, and 90%. Missing data may not contribute to percentiles. The counts may be performed by counting the number of rows in the data for a given variable for each patient.

Referring still to FIGS. 3A-3C, and in the embodiments described herein, it has been further noted that machine-learning algorithms and/or models may be used to analyze collected data. As an example, these models may be evaluated using scikit-learn below:
Random Forest (scikit-learn RandomForestClassifier);
Neural Network (scikit-learn MLPClassifier);
XGBoost;
Bagging (BalancedBaggingClassifier);
Linear SVM (scikit-learn SGDClassifier);
Logistic Regression (scikit-learn LogisticRegression);

In these embodiments, models may be compared using area under the ROC curve (AUC), sensitivity, and specificity on a 3-fold cross-validation. In some embodiments, hyperparameter tuning may be not performed due to the small number of positive samples in some subcategories. Hyperparameter tuning may be performed with sufficient number of samples in respective subcategories.

Further, as indicated above, Shapley Additive exPlanations (SHAP) analysis may be reviewed for IH+SIMV, IH, and SIMV models of the population of all infants as well as the subgroups of infants <2 weeks and ≥2 weeks of age. It should be appreciated that SHAP analyses assign a different weight to the top performing features for a model's prediction.

In some embodiments, various models may be beneficial for analyzing a variety of metrics described herein. For example, as shown in FIGS. 3A-3C, Random Forest analyses may perform best for the first model created using both data sets (IH+SIMV). Further, in some embodiments, analyzing infants partitioned by age increased accuracy: infants <2 weeks of age with extubation failure may predict with an AUC of 0.94, and infants ≥2 weeks of age may have their outcome predicted using XGBoost with an AUC of 0.83.

In the embodiments described herein, while any number of machine-learning models may be utilized, Random Forest may provide the most consistent performance as indicated by the highest average AUC throughout data sources and population analyses. SHAP analyses using Random Forest may be performed for all three models, and provided the following results:

For example, FIG. 3A depicts the IH+SIMV model. In these embodiments, for all ages, measured inhaled and expired tidal volume, and dynamic compliance may be the best performing features. For infants <2 weeks of age, the spontaneous and mandatory tidal volumes, resistance, and dynamic compliance may be the most influential features. For infants 2 weeks of age, the FiO₂, inhaled mandatory tidal volume, resistance, and minimum airway pressure may be the best performing features.

FIG. 3B depicts the IH alone mode. In these embodiments all ages, weight at extubation, birth weight, IH with SpO₂<80%, heart rate, gestational age, and age at extubation may be the most influential features. For infants <2 weeks of age, there may be multiple highly weighted features including birth weight, weight at extubation, GA, 5 minute Apgar, weight percentile at birth, age at extubation, heart rate, and percent time with SpO₂<80%. For infants 2 weeks of age, weight at extubation, gestational age, heart rate, IH events and percent time with SpO₂<80%, and age at extubation may be the most influential features.

FIG. 3C depicts the SIMV alone model. In these embodiments, for all ages, measured mandatory tidal volume and measured spontaneous tidal volume may be the most influential features. For infants <2 weeks of age, measured and mandatory tidal volume may be similarly the most influential features. For infants ≥2 weeks of age, FiO₂, time constant, spontaneous inspiratory and mandatory tidal volumes may be the most influential features. Other features may be more influential based on lung pathology or postnatal age for examples, pressure, volume, oxygen supplementation, and lung mechanics. Still some features may be more influential based on ventilator type (conventional versus high frequency) or mode.

The results depicted in FIGS. 3A-3C may be further illustrated in the following table:

TABLE 2
Top performing models by data source and subset population.

| Data source | Population | n | Algorithm | AUC | Sens. | Spec. | PPV | NPV |
|---|---|---|---|---|---|---|---|---|
| IH + SIMV | All | 65 | Random Forest | 0.77 | 0.59 | 0.91 | 0.74 | 0.84 |
| | Age <2 wks | 24 | Random Forest | 0.94 | 0.78 | 0.85 | 0.78 | 0.86 |
| | Age ≥2 wks | 41 | XGBoost | 0.83 | 0.42 | 0.97 | 0.83 | 0.83 |
| IH | All | 73 | SGDClassifier | 0.64 | 0.47 | 0.81 | 0.50 | 0.80 |
| | Age <2 wks | 27 | XGBoost | 0.77 | 0.56 | 0.72 | 0.42 | 0.79 |
| | Age ≥2 wks | 46 | XGBoost | 0.73 | 0.17 | 0.91 | 0.33 | 0.76 |
| SIMV | All | 100 | Random Forest | 0.71 | 0.13 | 0.88 | 0.31 | 0.75 |
| | Age <2 wks | 51 | Bagging | 0.86 | 0.80 | 0.78 | 0.60 | 0.90 |
| | Age ≥2 wks | 49 | XGBoost | 0.70 | 0.36 | 0.92 | 0.75 | 0.83 |

n: number of extubation events analyzed.
AUC: area under the ROC curve.
Sens.: sensitivity.
Spec.: specificity.
PPV: positive predictive value.
NPV: negative predictive value.
IH: intermittent hypoxemia.
SIMV: synchronized intermittent mandatory ventilation.

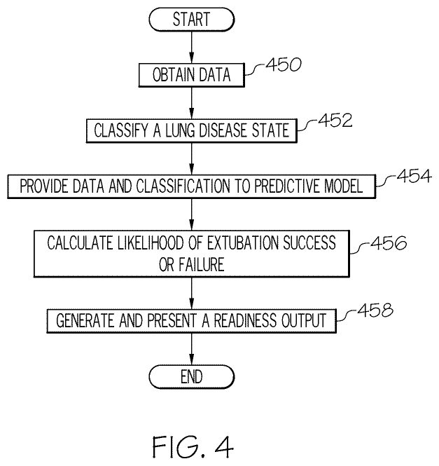

FIG. 4 depicts a flowchart for predicting extubation readiness in patients, according to embodiments provided herein. As illustrated in block 450, pulse oximeter data from the oximeter, monitor, and ventilator data from the ventilator may be obtained over a predetermined monitoring period. The pulse oximeter data may include at least oxygen saturation measurements indicative of intermittent hypoxemia events, heart rate measurements, perfusion measurements, etc. The ventilator data may include at least airway pressure rate, frequency, oxygen, lung mechanics, and ventilation mode. In block 452, a lung disease state of the preterm infant may be classified as acute or chronic based on at least one of the following: postnatal age and/or a clinical indicator. In block 454, the pulse oximeter data, ventilator data, monitor data, and the lung disease classification may be provided to a trained predictive model previously trained on training data from multiple infants. The training data may include oxygen saturation measurements indicative of intermittent hypoxemia events, heart rate measurements, ventilator data comprising tidal volume, airway pressure, lung mechanics, rate, frequency, and ventilation mode, and postnatal age and corresponding disease classification.

In block 456, a likelihood of extubation success or failure may be calculated within a clinically relevant time window, and generating at least one of the following: a positive predictive value (PPV) or a negative predictive value (NPV). In block 458, a readiness output based on the likelihood of extubation success or failure to facilitate extubation timing decisions by a clinical team may be generated and presented.

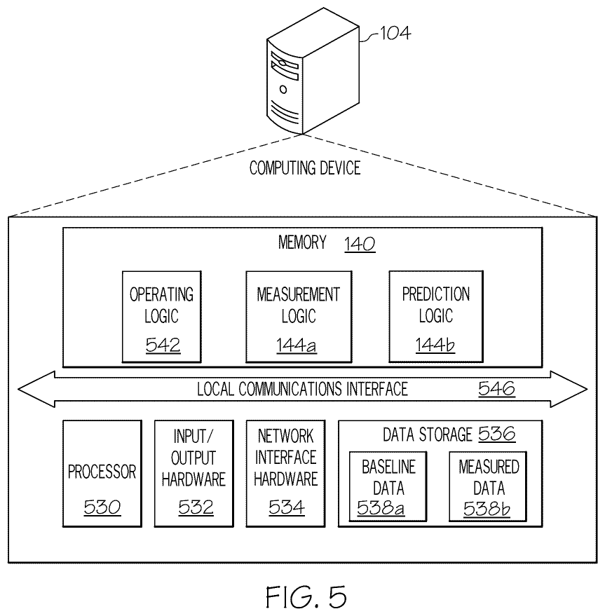

FIG. 5 depicts the computing device 104 for predicting extubation readiness in preterm infants, according to embodiments provided herein. As illustrated, the computing device 104 includes a processor 530, input/output hardware 532, a network interface hardware 534, a data storage component 536 (which stores baseline data 538a, measured data 538b, and/or other data, as described above), and a memory component 140. The memory component 140 may be configured as volatile and/or nonvolatile memory and as such, may include random access memory (including SRAM, DRAM, and/or other types of RAM), flash memory, secure digital (SD) memory, registers, compact discs (CD), digital versatile discs (DVD) (whether local or cloud-based), and/or other types of non-transitory computer-readable medium. Depending on the particular embodiment, these non-transitory computer-readable mediums may reside within the computing device 104 and/or external to the computing device 104.

The memory component 140 may store operating logic 542, the measurement logic 144a, and the prediction logic 144b, which may include the machine-learning model. Each of these logic components may include a plurality of different pieces of logic, each of which may be embodied as a computer program, firmware, and/or hardware, as an example. A local communication interface 546 is also included in FIG. 5 and may be implemented as a bus or other communication interface to facilitate communication among the components of the computing device 104.

The processor 530 may include any processing component operable to receive and execute instructions (such as from a data storage component 536 and/or the memory component 140). As described above, the input/output hardware 532 may include and/or be configured to interface with speakers, microphones, and/or other input/output components.

The network interface hardware 534 may include and/or be configured for communicating with any wired or wireless networking hardware, including an antenna, a modem, a LAN port, wireless fidelity (Wi-Fi) card, WiMAX card, mobile communications hardware, and/or other hardware for communicating with other networks and/or devices. From this connection, communication may be facilitated between the computing device 104 and other computing devices.

The operating logic 542 may include an operating system and/or other software for managing components of the computing device 104. As discussed above, the measurement logic 144a may reside in the memory component 140 and may be configured to cause the processor 530 to receive and process measurements from the medical device 102 (FIG. 1). The prediction logic 144b may include the machine-learning module and may be configured for causing a computing device (such as the computing device 104) to determining probabilities for success and/or failure of intubation and/or extubation, as described herein.

It should be understood that while the components in FIG. 5 are illustrated as residing within the computing device 104, this is merely an example. In some embodiments, one or more of the components may reside external to the computing device 104 or within other devices, such as the medical device 102 depicted in FIG. 1. It should also be understood that, while the computing device 104 is illustrated as a single device, this is also merely an example. In some embodiments, the measurement logic 144a and the prediction logic 144b may reside on different computing devices.

As an example, one or more of the functionalities and/or components described herein may be provided by the computing device 104, and/or other computing device. Depending on the particular embodiment, any of these devices may have similar components as those depicted in FIG. 5. To this end, any of these devices may include logic for performing the functionality described herein.

Additionally, while the computing device 104 is illustrated with the measurement logic 144a and the prediction logic 144b as separate logical components, this is also an example. In some embodiments, a single piece of logic may provide the described functionality. It should also be understood that while the measurement logic 144a and the prediction logic 144b are described herein as the logical components, this is also an example. Other components may also be included, depending on the embodiment.

As should be appreciated in view of the foregoing, embodiments for predicting extubation readiness in preterm infants utilizing combined pulse oximeter and bedside ventilator data may utilize a machine-learning model IH+SIMV achieving an AUC of 0.94 for extubation failure in the first 2 weeks of life and AUC of 0.83 after 2 weeks of life. This predictive ability is comparable to or higher than previously reported machine-learning based extubation readiness protocols.

In addition to the high predictive ability of these embodiments, a clinically relevant aspect is the data source. These embodiments utilize streams of data from the bedside pulse oximeters, monitors, and/or ventilators. These sources of data are currently available for clinical teams in NICUs as well as other intensive care settings that manage patients requiring ventilation. Moreover, these readily available resources increase the feasibility to implement these embodiments in contrast to current solutions that utilize specialized devices in assessing extubation readiness. These embodiments may separate and combine sets of pulse oximeter and ventilator data with clinically relevant AUC, NPV, and/or PPV to make it possible for implementation across many medical settings with varying equipment.

Another aspect is the inclusion of not only variables set by a clinical team (e.g. PEEP, rate), but also measured variables from the pulse oximeter and ventilator which correlate to the changing clinical status of the patients, such as measured tidal volume, compliance, and IH events, heart rate, perfusion indices, respiratory rate, frequency, oxygen needs. These measured variables may be viewed as more reflective of the patients' respiratory condition, which may strengthen our model prediction. For example, the ventilator settings as determined by the medical team are less represented in the most heavily weighted features. This is of interest as ventilator settings are prominently featured in most institutions' extubation readiness criteria.

As described in detail herein, “failure” may refer to reintubation within seven days instead of 48 to 72 hours or within two days, three days, four days, etc. It should be appreciated that defining extubation failure at 48-72 hours may overestimate extubation success as many infants fail between days 3-7. On the other hand, after seven days, most reintubation reasons are of non-pulmonary in origin and may overestimate extubation failure. Extubation failure within 7 days, as defined herein, may be the best outcome measure for extubation readiness in this patient population. Further, in some embodiments, it should be appreciated that failure may be defined as reintubation within any period of time (e.g., minutes, hours, days, etc.) without departing from the scope of the present disclosure.

The disclosed embodiments may also differ from current solutions that select first time or elective extubation events only. While inclusion of any extubation occurrence has potential to add noise to the result, it broadens the population, on which the model can be applied, making it more practical. Secondly, the ventilator analysis may include only conventional modes of ventilation and excludes high frequency modes. The partition of infants at 2 weeks of age may be decided with consensus agreement to differentiate acute perinatal lung conditions such as respiratory distress syndrome from evolving chronic lung disease. However, these may apply to other modalities of ventilation including high frequency jet ventilation, high frequency oscillatory ventilation, and other conventional modes.

It should be further appreciated that the disclosed embodiments may focus analyses on ventilator and pulse oximetry continuous data that reflects the changing physiological status of the infant immediately prior to extubation. This bedside data may be collected directly from the ventilators and/or bedside monitors.

Some embodiments provided herein utilize machine-learning or other analyses techniques to handle large streams of data to produce prediction models for extubation failure or success. Using machine-learning, the method and system allow for enhanced accuracy for predicting extubation failure in preterm infants by utilizing combined data streams from pulse oximeters and ventilators. These algorithms may be further developed to become applicable in other NICU or medical settings with varied ventilator and bedside monitor types.

It should be further appreciated that the disclosed embodiments may focus on conventional ventilation, but the method and system for enhanced accuracy for predicting extubation failure or success may be further developed to predict impending respiratory failure and need for intubation or advanced respiratory support of a patient who is not currently intubated by utilizing the data streams as described in this document. The data streams can be non-invasive respiratory support devices (either synchronized or non-synchronized) and/or bedside monitoring

It should be further appreciated that the disclosed embodiments may be further developed to be used for titrating, weaning, or increasing respiratory support in patients on the ventilator regardless of type (e.g. conventional, high frequency ventilator, non-invasive respiratory support).

While some embodiments provided herein focus on human preterm infants, embodiments provided herein are contemplated for term infants, children, and adults. While the underlying physiology and pathology of these human patient populations may differ somewhat, the machine learning and analyses algorithms are configured to take into account the age of the patient and lung pathology.

Similarly, while some embodiments focus on human patients, embodiments provided herein may be configured for non-human mammals. Medical studies of disease states or toxic exposures in animal models, especially mammals, is well established because of the similarities between other mammalian pulmonary systems and humans. For example, tuberculosis has been studied in non-human primates, pigs, rabbits, and rodents and, more directly, established premature lung and IH models include rats, lambs, and baboons. Similarly, in the medical care of animals as performed within the veterinarian sciences, ventilators are commonly employed in cases of transient respiratory failure as is performed in human medicine. Veterinarian medical providers must also assess the extubation readiness status of their animal patients. There is overlapping physiology of mammals' pulmonic systems, similarities of the ventilator settings used in veterinarian care, for example, tidal volume, rate, PEEP, fraction inspired oxygen, as well as the use of monitoring devices, for example pulse oximetry. The method and system may be applied to assess risk for extubation success or failure and respiratory management in the non-human mammal within both scientific study and veterinary medicine.

Accordingly, a first aspect of this disclosure includes a method for predicting extubation readiness for a patient comprising: obtaining, by a computing device, pulse oximeter data and ventilator data for a patient that has been intubated over a predetermined monitoring period, wherein the pulse oximeter data includes at least one of the following: oxygen saturation measurements indicative of intermittent hypoxemia events, heart rate measurements, or perfusion, and the ventilator data includes at least one of the following: volume, pressure, a ventilation mode, rate, frequency, oxygen supplementation, inspiratory times, expiratory times, or lung mechanics; classifying, by the computing device, a lung disease state of the patient as acute or chronic based on at least one of the following: an age of the patient or a clinical indicator; providing, by the computing device, the pulse oximeter data, the ventilator data, and a lung disease classification to a trained predictive model previously trained on training data, wherein the training data includes at least one of the following: oxygen saturation measurements indicative of intermittent hypoxemia events, heart rate measurements, or perfusion, the ventilator data including at least one of the following: volume, pressure, a ventilation mode, rate, frequency, oxygen supplementation, inspiratory times, expiratory times, or lung mechanics; calculating, by the computing device, a likelihood of extubation success or failure within a clinically relevant time window, and generating at least one of the following: a positive predictive value (PPV) or a negative predictive value (NPV); and generating and presenting, by the computing device, a readiness output based on the likelihood of extubation success or failure for determining extubation timing.

A second aspect includes the first aspect, further comprising selecting a 2-hour predictive interval within a monitoring period for calculating the likelihood of extubation success or failure.

A third aspect includes the first and/or second aspect, further comprising outputting a success likelihood or a failure likelihood that applies to a time interval following a potential extubation event.

A fourth aspect includes any of the first aspect through the third aspect, further comprising determining differing probabilities of extubation success or failure and presenting the readiness output corresponding to the differing probabilities of extubation success or failure.

A fifth aspect includes any of the first aspect through the fourth aspect, wherein presenting the readiness output includes providing distinct outputs for the following: a first indicator for a high probability of extubation success; a second indicator for an intermediate or uncertain probability; and a third indicator for a high probability of extubation failure.

A sixth aspect includes any of the first aspect through the fifth aspect, wherein the trained predictive model assigns distinct weighting factors to the ventilator data based on an identified ventilation mode, including at least one weight for conventional ventilation modes and a different weight for high-frequency modes.

A seventh aspect includes any of the first aspect through the sixth aspect, wherein classifying the lung disease state further comprises differentiating among at least one of the following: acute neonatal respiratory distress syndrome, evolving chronic lung disease, established bronchopulmonary dysplasia, infection, restrictive or obstructive lung diseases, cardiac congenital disease, or pulmonary congenital disease, and wherein the method further comprises transitioning the lung disease classification of the lung disease state from acute to chronic based on at least one of the following: a predetermined postnatal age threshold or a detected change in measured lung dynamics and mechanics that is beyond a predefined range.

An eighth aspect includes any of the first aspect through the seventh aspect, further comprising generating an extubation alert when the likelihood of success or failure surpasses a predetermined threshold, wherein the extubation alert is transmitted to the computing device.

A ninth aspect includes any of the first aspect through the eighth aspect, wherein intermittent hypoxemia events include a period in which an oxygen saturation falls below a predetermined threshold for at least a determined period of time.

A tenth aspect includes any of the first aspect through the ninth aspect, preprocessing the pulse oximeter data and the ventilator data to align sampling intervals.

An eleventh aspect includes any of the first aspect through the tenth aspect, further comprising determining a likelihood of success or failure of reintubating the patient.

A twelfth aspect includes a system for predicting extubation readiness for a patient comprising: an oximeter for measuring an oxygen level of a patient; a ventilator for intubating and ventilating the patient; and a computing device that includes a memory component and logic, wherein when the logic is executed by the computing device, the logic causes the system to perform at least the following: obtain pulse oximeter data from the oximeter and ventilator data from the ventilator for the patient that has been intubated over a predetermined monitoring period, wherein the pulse oximeter data includes at least one of the following: oxygen saturation measurements indicative of intermittent hypoxemia events, heart rate measurements, or perfusion, and the ventilator data includes at least one of the following: volume, pressure, a ventilation mode, rate, frequency, oxygen supplementation, inspiratory times, expiratory times, or lung mechanics; classify a lung disease state of the patient as acute or chronic based on at least one of the following: an age of the patient or a clinical indicator; provide the pulse oximeter data, the ventilator data, and a lung disease classification to a trained predictive model previously trained on training data, wherein the training data includes at least one of the following: oxygen saturation measurements indicative of intermittent hypoxemia events, heart rate measurements, or perfusion, the ventilator data including at least one of the following: volume, pressure, a ventilation mode, rate, frequency, oxygen supplementation, inspiratory times, expiratory times, or lung mechanics; calculate a likelihood of extubation success or failure within a clinically relevant time window, and generating at least one of the following: a positive predictive value (PPV) or a negative predictive value (NPV); and generate and present a readiness output based on the likelihood of extubation success or failure for determining extubation timing.

A thirteenth aspect includes the twelfth aspect, wherein the logic further causes the system to perform at least the following: select a predetermined predictive interval within a monitoring period for calculating the likelihood of extubation success or failure; and output a success likelihood or a failure likelihood that applies to a time interval following a potential extubation event.

A fourteenth aspect includes the twelfth aspect and/or the thirteenth aspect, wherein the logic further causes the system to determine differing probabilities of extubation success and present the readiness output corresponding to the differing probabilities of extubation success or failure and wherein presenting the readiness output includes providing distinct outputs for the following: a first indicator for a high probability of extubation success; a second indicator for an intermediate or uncertain probability; and a third indicator for a high probability of extubation failure.

A fifteenth aspect includes any of the twelfth aspect through the fourteenth aspect, wherein the trained predictive model assigns distinct weighting factors to the ventilator data based on an identified ventilation mode, including at least one weight for conventional ventilation modes and a different weight for high-frequency modes.

A sixteenth aspect includes any of the twelfth aspect through the fifteenth aspect, wherein classifying the lung disease state further comprises differentiating among at least the following: acute neonatal respiratory distress syndrome, evolving chronic lung disease, and established bronchopulmonary dysplasia.

A seventeenth aspect includes any of the twelfth aspect through the sixteenth aspect, further comprising a remote computing device wherein the logic further causes the system to generate an extubation alert when the likelihood of success or failure surpasses a predetermined threshold, wherein the extubation alert is transmitted to the remote computing device.

An eighteenth aspect includes any of the twelfth aspect through the seventeenth aspect, wherein intermittent hypoxemia events include a period in which an oxygen saturation falls below a predetermined threshold for at least a determined period of time.

A nineteenth aspect includes a medical device for predicting extubation readiness for a patient that includes a display and stores logic that, when executed by the medical device, causes the medical device to perform at least the following: obtain pulse oximeter data and ventilator data for a patient that has been intubated over a predetermined monitoring period, wherein the pulse oximeter data includes at least one of the following: oxygen saturation measurements indicative of intermittent hypoxemia events, heart rate measurements, or perfusion, and the ventilator data includes at least one of the following: volume, pressure, a ventilation mode, rate, frequency, oxygen supplementation, inspiratory times, expiratory times, or lung mechanics; classify a lung disease state of the patient as acute or chronic based on at least one of the following: an age of the patient or a clinical indicator; provide the pulse oximeter data, the ventilator data, and a lung disease classification to a trained predictive model previously trained on training data, wherein the training data includes at least one of the following: oxygen saturation measurements indicative of intermittent hypoxemia events, heart rate measurements, or perfusion, the ventilator data including at least one of the following: volume, pressure, a ventilation mode, rate, frequency, oxygen supplementation, inspiratory times, expiratory times, or lung mechanics; calculate a likelihood of extubation success or failure within a clinically relevant time window, and generating at least one of the following: a positive predictive value (PPV) or a negative predictive value (NPV); and generate and present a readiness output via the display based on the likelihood of extubation success or failure for determining extubation timing.

A twentieth aspect includes the nineteenth aspect, wherein the logic further causes the medical device to determine differing probabilities of extubation success or failure and present the readiness output corresponding to the differing probabilities of extubation success or failure and wherein presenting the readiness output includes providing distinct outputs for the following: a first indicator for a high probability of extubation success; a second indicator for an intermediate or uncertain probability; and a third indicator for a high probability of extubation failure.

While particular embodiments and aspects of the present disclosure have been illustrated and described herein, various other changes and modifications can be made without departing from the spirit and scope of the disclosure. Moreover, although various aspects have been described herein, such aspects need not be utilized in combination. Accordingly, it is therefore intended that the appended claims cover all such changes and modifications that are within the scope of the embodiments shown and described herein.

It should now be understood that embodiments disclosed herein include systems, devices, and methods for automatically determining extubation readiness. It should also be understood that these embodiments are merely exemplary and are not intended to limit the scope of this disclosure.

What is claimed is:

1. A method for predicting extubation readiness for a patient comprising:
obtaining, by a computing device, pulse oximeter data and ventilator data for a patient that has been intubated over a predetermined monitoring period, wherein the pulse oximeter data includes at least one of the following: oxygen saturation measurements indicative of intermittent hypoxemia events, heart rate measurements, or perfusion, and the ventilator data includes at least one of the following: volume, pressure, a ventilation mode, rate, frequency, oxygen supplementation, inspiratory times, expiratory times, or lung mechanics;
classifying, by the computing device, a lung disease state of the patient as acute or chronic based on at least one of the following: an age of the patient or a clinical indicator;
providing, by the computing device, the pulse oximeter data, the ventilator data, and a lung disease classification to a trained predictive model previously trained on training data, wherein the training data includes at least one of the following: oxygen saturation measurements indicative of intermittent hypoxemia events, heart rate measurements, or perfusion, the ventilator data including at least one of the following: volume, pressure, a ventilation mode, rate, frequency, oxygen supplementation, inspiratory times, expiratory times, or lung mechanics;
calculating, by the computing device, a likelihood of extubation success or failure within a clinically relevant time window, and generating at least one of the following: a positive predictive value (PPV) or a negative predictive value (NPV); and
generating and presenting, by the computing device, a readiness output based on the likelihood of extubation success or failure for determining extubation timing.

2. The method of claim 1, further comprising selecting a 2-hour predictive interval within a monitoring period for calculating the likelihood of extubation success or failure.

3. The method of claim 2, further comprising outputting a success likelihood or a failure likelihood that applies to a time interval following a potential extubation event.

4. The method of claim 1, further comprising determining differing probabilities of extubation success or failure and presenting the readiness output corresponding to the differing probabilities of extubation success or failure.

5. The method of claim 4, wherein presenting the readiness output includes providing distinct outputs for the following:
a first indicator for a high probability of extubation success;
a second indicator for an intermediate or uncertain probability; and
a third indicator for a high probability of extubation failure.

6. The method of claim 1, wherein the trained predictive model assigns distinct weighting factors to the ventilator data based on an identified ventilation mode, including at least one weight for conventional ventilation modes and a different weight for high-frequency modes.

7. The method of claim 1, wherein classifying the lung disease state further comprises differentiating among at least one of the following: acute neonatal respiratory distress syndrome, evolving chronic lung disease, established bronchopulmonary dysplasia, infection, restrictive or obstructive lung diseases, cardiac congenital disease, or pulmonary congenital disease, and wherein the method further comprises transitioning the lung disease classification of the lung disease state from acute to chronic based on at least one of the following: a predetermined postnatal age threshold or a detected change in measured lung dynamics and mechanics that is beyond a predefined range.

8. The method of claim 1, further comprising generating an extubation alert when the likelihood of success or failure surpasses a predetermined threshold, wherein the extubation alert is transmitted to the computing device.

9. The method of claim 1, wherein intermittent hypoxemia events include a period in which an oxygen saturation falls below a predetermined threshold for at least a determined period of time.

10. The method of claim 1, preprocessing the pulse oximeter data and the ventilator data to align sampling intervals.

11. The method of claim 1, further comprising determining a likelihood of success or failure of reintubating the patient.

12. A system for predicting extubation readiness for a patient comprising:
an oximeter for measuring an oxygen level of a patient;
a ventilator for intubating and ventilating the patient; and
a computing device that includes a memory component and logic, wherein when the logic is executed by the computing device, the logic causes the system to perform at least the following:
obtain pulse oximeter data from the oximeter and ventilator data from the ventilator for the patient that has been intubated over a predetermined monitoring period, wherein the pulse oximeter data includes at least one of the following: oxygen saturation measurements indicative of intermittent hypoxemia events, heart rate measurements, or perfusion, and the ventilator data includes at least one of the following: volume, pressure, a ventilation mode, rate, frequency, oxygen supplementation, inspiratory times, expiratory times, or lung mechanics;
classify a lung disease state of the patient as acute or chronic based on at least one of the following: an age of the patient or a clinical indicator;
provide the pulse oximeter data, the ventilator data, and a lung disease classification to a trained predictive model previously trained on training data, wherein the training data includes at least one of the following: oxygen saturation measurements indicative of intermittent hypoxemia events, heart rate measurements, or perfusion, the ventilator data including at least one of the following: volume, pressure, a ventilation mode, rate, frequency, oxygen supplementation, inspiratory times, expiratory times, or lung mechanics;
calculate a likelihood of extubation success or failure within a clinically relevant time window, and generating at least one of the following: a positive predictive value (PPV) or a negative predictive value (NPV); and
generate and present a readiness output based on the likelihood of extubation success or failure for determining extubation timing.

13. The system of claim 12, wherein the logic further causes the system to perform at least the following:
select a predetermined predictive interval within a monitoring period for calculating the likelihood of extubation success or failure; and
output a success likelihood or a failure likelihood that applies to a time interval following a potential extubation event.

14. The system of claim 12, wherein the logic further causes the system to determine differing probabilities of extubation success or failure and present the readiness output corresponding to the differing probabilities of extubation success and wherein presenting the readiness output includes providing distinct outputs for the following:
a first indicator for a high probability of extubation success;
a second indicator for an intermediate or uncertain probability; and
a third indicator for a high probability of extubation failure.

15. The system of claim 12, wherein the trained predictive model assigns distinct weighting factors to the ventilator data based on an identified ventilation mode, including at least one weight for conventional ventilation modes and a different weight for high-frequency modes.

16. The system of claim 12, wherein classifying the lung disease state further comprises differentiating among at least the following:
acute neonatal respiratory distress syndrome,
evolving chronic lung disease, and
established bronchopulmonary dysplasia.

17. The system of claim 12, further comprising a remote computing device wherein the logic further causes the system to generate an extubation alert when the likelihood of success or failure surpasses a predetermined threshold, wherein the extubation alert is transmitted to the remote computing device.

18. The system of claim 12, wherein intermittent hypoxemia events include a period in which an oxygen saturation falls below a predetermined threshold for at least a determined period of time.

19. A medical device for predicting extubation readiness for a patient that includes a display and stores logic that, when executed by the medical device, causes the medical device to perform at least the following:
obtain pulse oximeter data and ventilator data for a patient that has been intubated over a predetermined monitoring period, wherein the pulse oximeter data includes at least one of the following: oxygen saturation measurements indicative of intermittent hypoxemia events, heart rate measurements, or perfusion, and the ventilator data includes at least one of the following: volume, pressure, a ventilation mode, rate, frequency, oxygen supplementation, inspiratory times, expiratory times, or lung mechanics;
classify a lung disease state of the patient as acute or chronic based on at least one of the following: an age of the patient or a clinical indicator;
provide the pulse oximeter data, the ventilator data, and a lung disease classification to a trained predictive model previously trained on training data, wherein the training data includes at least one of the following: oxygen saturation measurements indicative of intermittent hypoxemia events, heart rate measurements, or perfusion, the ventilator data including at least one of the following: volume, pressure, a ventilation mode, rate, frequency, oxygen supplementation, inspiratory times, expiratory times, or lung mechanics;
calculate a likelihood of extubation success or failure within a clinically relevant time window, and generating at least one of the following: a positive predictive value (PPV) or a negative predictive value (NPV); and
generate and present a readiness output via the display based on the likelihood of extubation success or failure for determining extubation timing.

20. The medical device of claim 19, wherein the logic further causes the medical device to determine differing probabilities of extubation success or failure and present the readiness output corresponding to the differing probabilities of extubation success or failure and wherein presenting the readiness output includes providing distinct outputs for the following:
a first indicator for a high probability of extubation success;
a second indicator for an intermediate or uncertain probability; and
a third indicator for a high probability of extubation failure.
